(19) United States
(12) Patent Application Publication
(10) Pub. No.: US 2021/0338529 A1
(43) Pub. Date: Nov. 4, 2021

(54) INTRA ORAL WEARABLE DEVICE AND A METHOD FOR DELIVERING FLUIDS

(71) Applicant: Stephen Snow, Queensland (AU)

(72) Inventor: Stephen Snow, Queensland (AU)

(21) Appl. No.: 16/865,712

(22) Filed: May 4, 2020

(51) Int. Cl. A61J 7/00; A61M 16/00

(52) U.S. Cl. CPC A61J 7/0015; A61M 2205/076; A61M 2210/0625; A61M 16/0057

(57) ABSTRACT

There is provided an intra oral wearable device and a method for delivering fluids. The wearable device comprises a central body adapted to be positioned in a mouth of a user, one or more tentacles connected at a periphery of the central body; and one or more inserts adapted to be inserted over the one or more tentacles to improve adherence and comfort. Further, the central body has a top layer, a bottom layer comprising a plurality of holes and a fluid housing enclosed between the top layer and the bottom layer. The fluid housing contains a fluid. The fluid may be one of nutraceutical and/or pharmaceutical fluids, re-hydration liquids or gels, nutritional liquids and oxygen. Further, the one or more inserts and the one or more tentacles are configured to be clamped to an upper jaw of the mouth of the user and secure the central body therein.

This application claims the benefit of U.S. Provisional Patent Application Ser. No. 62/831,349 filed Apr. 16, 2019, which is expressly incorporated by reference herein. The delay in filing the nonprovisional application within the twelve-month period set forth in 37 CFR 1.78(a)(1)(i) was unintentional. Applicant submits hereto its Statement of Delay due to COVID-19 Outbreak.

### TECHNICAL FIELD

The present disclosure, generally, relates to physical health care and oral fluid delivery systems and more particularly relates to an intra oral wearable device and a method for delivering fluids.

### BACKGROUND

Dehydration has always been a problem for people especially during physical activities. For athletes, it is even more important to stay hydrated. During the physical activities, when people, especially athletes are giving their best and pushing their body to the limit, they also tend to lose a lot of fluid that can cause a lot of health problems in the short and long term. There is also a possibility of critical nutrient depletion in the body during activities such as sports and athletics. Abnormal heartbeat, blood pressure and breathing problems are some other bad effects of the dehydration. Also, water is involved in the majority of chemical reactions involved in athletic performance. It is important that athletes are hydrated before, during and after physical activity to achieve their maximal physical performance. Otherwise, a dehydrated athlete with 2.5% loss of body weight in the form of water can experience up to a 45% loss in the capacity to perform high-intensity exercise. This is because the dehydration can cause reduction in blood volume, decreased sweat rate, decreased heat distribution and increased rate of muscle glycogen use. These problems leave the athletes prone to cardiac arrest, heat stroke, skeletal muscle fatigue etc.

Presently only solution available for people for above mentioned problems is oral rehydration solutions, rehydration liquids that people can drink when they feel dehydrated. For athletes, the problem is bigger because they only have access to such drinks during their scheduled time-outs or breaks. These drinks are generally administered through bottles and shakers when the athletes take a scheduled or a forced break. But the problem is that there is no solution available for the athletes to stay hydrated during the game. There are no active solutions available to protect the athletes to prevent any health problems from happening during the game. There have already been a lot of incidents around the world where players have collapsed for example, during football matches in Asian and African countries, there were forced drink breaks due to hot environmental conditions. The danger becomes even more alarming with the ongoing global warming phenomenon.

Therefore, there is a need in the art for an intra oral wearable device and a method for delivering fluids which ameliorates all or some of the deficiencies of the prior art and provides a viable and effective alternative.

### SUMMARY

According to a first aspect of the present disclosure, there is provided an intra oral wearable device for delivering fluids. The wearable device comprises a central body adapted to be positioned in mouth of a user, the central body having a top layer, a bottom layer comprising a plurality of holes and a fluid housing enclosed between the top layer and the bottom layer, the fluid housing containing a fluid; one or more tentacles connected at a periphery of the central body; and one or more inserts adapted to be inserted over the one or more tentacles to improve adherence and comfort. Further, the one or more inserts and the one or more tentacles are configured to be clamped to an upper jaw of the mouth of the user and secure the central body therein. Furthermore, after positioning the central body in the mouth, the top layer is proximal to a palate inside the mouth and the bottom layer is proximal to a tongue of the user. Additionally, the bottom layer is adapted to be pressed by the tongue that consequently squeezes the fluid housing to secrete the fluid stored therein. Next, the fluid secreted above is delivered to the mouth of the user through the plurality of holes in the bottom layer.

In accordance with an embodiment of the present invention, the central body and the tentacles are an integral unit.

In accordance with an embodiment of the present invention, the central body, the one or more tentacles and the one or more inserts are made of silicone.

In accordance with an embodiment of the present invention, the fluid is selected from nutraceutical fluids, pharmaceutical fluids, re-hydration liquids or gels, nutritional liquids and oxygen.

According to a first aspect of the present disclosure, there is provided a method for delivering fluids intra orally. The method comprising the steps of positioning a central body of a wearable device in a mouth of a user by clamping one or more tentacles to an upper jaw of teeth; pressing a bottom layer of the central body by a tongue and consequently squeezing a fluid housing to secrete a fluid stored therein; and delivering the fluid secreted to the mouth of the user through a plurality of holes in the bottom layer.

### BRIEF DESCRIPTION OF THE DRAWINGS

So that the manner in which the above recited features of the present disclosure can be understood in detail, a more particular description of the disclosure, briefly summarized above, may have been referred by embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

These and other features, benefits, and advantages of the present disclosure will become apparent by reference to the following text figure, with like reference numbers referring to like structures across the views, wherein:

FIG. 1A-1B illustrate an intra oral wearable device for delivering fluids, in accordance with an embodiment of the present invention;

FIG. 1C illustrates an intra oral wearable device for delivering fluids, in accordance with another embodiment of the present invention; and

FIG. 2A-2C illustrates an implementation of the intra oral wearable device of FIG. 1A-1B, in accordance with an embodiment of the present invention.

### DETAILED DESCRIPTION OF THE EMBODIMENTS

While the present invention is described herein by way of example using embodiments and illustrative drawings, those skilled in the art will recognize that the invention is not limited to the embodiments of drawing or drawings described, and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claim. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes.

The present invention is described hereinafter by various embodiments with reference to the accompanying drawings, wherein reference numerals used in the accompanying drawing correspond to the like elements throughout the description. This invention may, however, be embodied in many different forms and should not be construed as limited to the embodiment set forth herein. Rather, the embodiment is provided so that this disclosure will be thorough and complete and will fully convey the scope of the invention to those skilled in the art. In the following detailed description, numeric values and ranges are provided for various aspects of the implementations described. These values and ranges are to be treated as examples only and are not intended to limit the scope of the claims. In addition, a number of materials are identified as suitable for various facets of the implementations. These materials are to be treated as exemplary and are not intended to limit the scope of the invention.

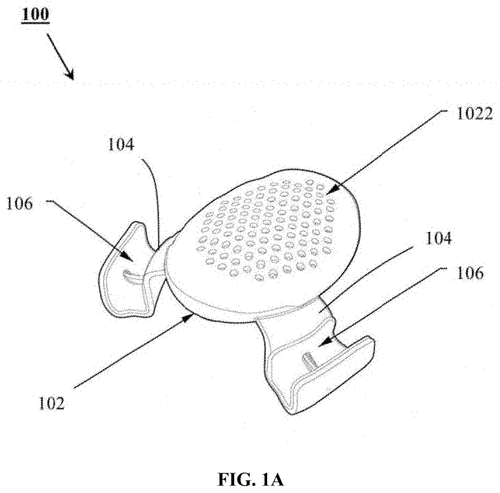

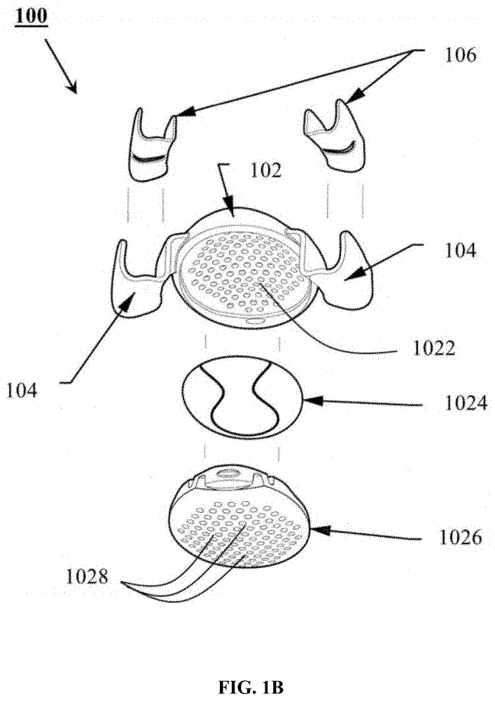

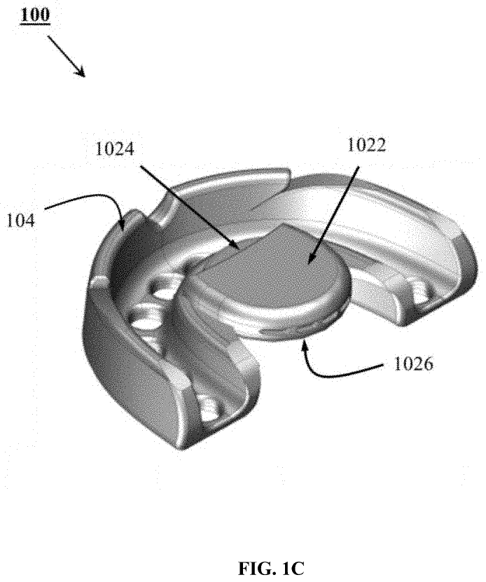

FIG. 1A-1B illustrates an intra oral wearable device (100) for delivering fluids, in accordance with an embodiment of the present invention. As shown in FIG. 1A, the intra oral wearable device (100) (hereinafter referred to as “the wearable device (100)”) is a mouth guard adapted to be placed in a mouth of a user (not shown in FIGS. 1A-1B). The mouth guard can be clamped to an upper jaw in the mouth of the user. The design of the wearable device (100) is more compact and lighter in weight than presently used mouth guards, therefore do not cause strain on the mouth or jaws. FIG. 1B illustrates an exploded view of the wearable device (100) of FIG. 1A, in accordance with an embodiment of the present invention. As shown in FIG. 1B, the wearable device (100) comprises a central body (102) adapted to be positioned in the mouth (not shown in FIGS. 1A-1B) of the user, one or more tentacles (104) connected at a periphery of the central body (102) and one or more inserts (106) adapted to be inserted over the one or more tentacles (104) to improve adherence and comfort.

The central body (102) further comprises a top layer (1022) and a bottom layer (1026). After positioning the central body (102) in the mouth, the top layer (1022) is proximal to a palate inside the mouth and the bottom layer (1026) is proximal to a tongue of the user. Further, a fluid housing (1024) is enclosed between the top layer (1022) and the bottom layer (1026). The fluid housing (1024) contains a fluid. Further, the fluid housing (1024) may be shaped as, but not limited to, a squeezable pouch adapted to release the fluid when squeezed or pressed.

The fluid may be selected from one of, but not limited to, nutraceutical fluids, pharmaceutical fluids, re-hydration liquids or gels, nutritional liquids and oxygen. The fluid may be in a form of a densely concentrated solution, a soluble solid, a gel or a gas. Furthermore, the bottom layer (1026) has a plurality of holes (1028) that allow the fluid housing (1024) to be in fluidic communication with the mouth. In one embodiment, the top layer (1022) may also have a plurality of holes so that palate may also come in contact with the fluid upon its release. Upon release, the fluid entering the mouth is mixed with saliva or water to be easily assimilated.

In accordance with an embodiment of the present invention, the fluid may be selected as per the requirements of the user. It will be appreciated by a person skilled in the art that the usage of present invention is not limited to athletes or for rehydration purposes only, but it can also be used as a source of nutrition for all kinds of users or even for emergency breathing assistance, without departing from the scope of the present invention. In that sense, nutritional liquids may be provided as fluid in the fluid housing (1024), if the wearable device (100) is to be used for providing nutritional drinks. In case the wearable device (100) is to be used as an emergency breathing assistance device, the oxygen may be provided as fluid in the fluid housing (1024).

Returning back to FIG. 1B, the one or more tentacles (104) are provided on the periphery of the central body (102). The one or more tentacles (104) are a kind of U-shaped receptacles that are adapted to conform over the upper jaw of the teeth. So, there may be a single tentacle covering a broader portion of upper jaw or there may be two or more tentacles covering smaller portions of the upper jaw, thereby being more comfortable. In the exemplary embodiment shown in FIGS. 1A-1B, there are two tentacles extending sideways reaching the upper jaw of teeth with function of fastening the wearable device (100) inside the mouth by clamping directly onto first and second set of premolars. Whereas, in the exemplary embodiment shown in FIG. 1C that illustrates another embodiment of the wearable device of the present invention. As shown in FIG. 1C, there is a single tentacle (104) covering the broader portion of upper jaw and configured to be clamped thereon.

The central body (102) and the one or more tentacles (104) may be manufactured by, but not limited to, moulding. The material may be, but not limited to, plastic or semi-rigid silicone or the any other flexible and user-friendly material. The one or more tentacles (104) may be designed in various sizes to cover different jaw structures as well as people of all ages irrespective of gender. In accordance with an embodiment of the present invention, the central body (102) and the one or more tentacles (104) are an integral unit and are moulded together.

Additionally, the one or more inserts (106) are provided to fit in the U-shaped structure of the one or more tentacles (104). The one or more inserts (106) enhance the adherence of the one or more tentacles (104) to the teeth and are more comfortable for the users once they are clamped to the teeth. The one or more inserts (106) are made of, but not limited to, silicone and their number is selected depending upon the number of tentacles present in the wearable device (100). In the another embodiment shown in FIG. 1C, there may not be requirement for one or more inserts (106) and the improved adherence and comfort of the one or more inserts (106) may be provided in the one or more tentacles (104).

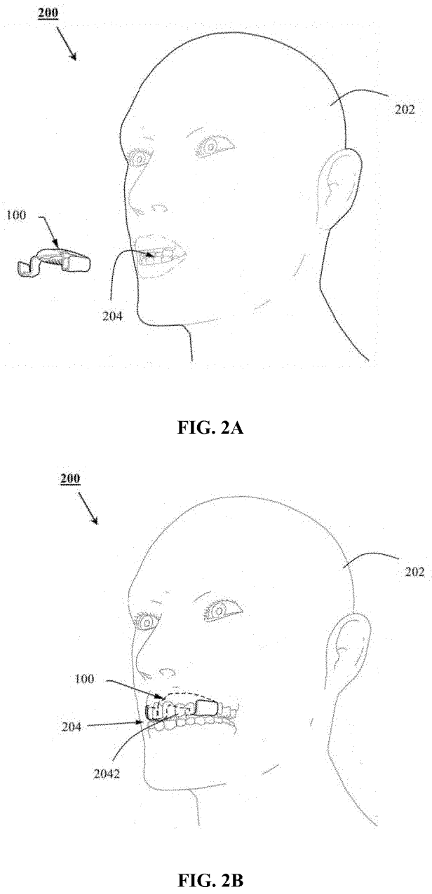

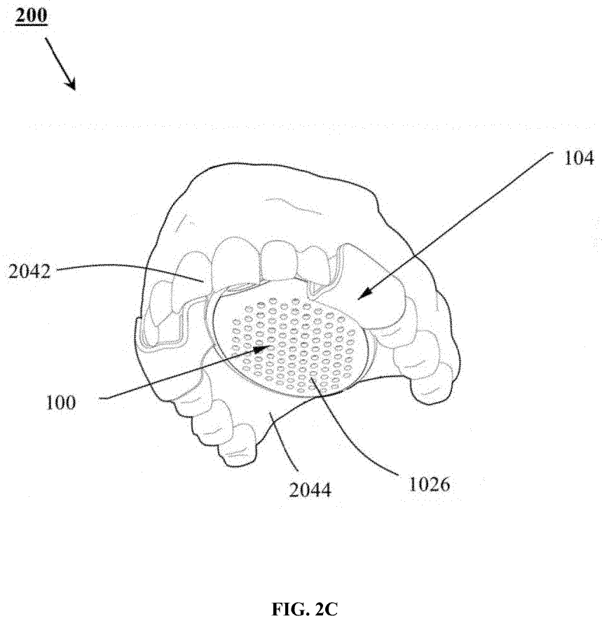

FIG. 2A-2C illustrate an implementation/method (200) of the wearable device (100), in accordance with an embodiment of the present invention. As shown in FIG. 2A and 2B, the wearable device (100) is inserted in the mouth (204) of the user (202). The one or more inserts (106) along with the one or more tentacles (104) are configured to be clamped to an upper jaw (2042) of the mouth (204) of the user (202) and secure the central body (102) therein. The same has been more clearly illustrated in FIG. 2C that illustrates a detailed view of the implementation. Inserting the wearable device (100) in the mouth (202) is similar to inserting any existing mouth guards except that they do not offer fluid delivery functionality and do not have a compact & comfortable design like that of the present invention.

For the sake of explanation, it is assumed that the user (202) is an athlete and the fluid stored in the fluid housing (1024) is a rehydration liquid. So, after the wearable device (100) is secured in the mouth (204) of the user (202), he/she may continue with his/her physical activity. For example: if the user (202) is a footballer or a swimmer, he/she may continue with his/her practice/game. Then, as the user (202) feels dehydrated after some time, he/she only requires pressing the bottom layer (1026) of the central body (102) with the tongue. Then, as the bottom layer (1026) is pressed, it consequently squeezes the fluid housing (1024) sandwiched between the bottom layer (1026) and the top layer (1022). This secretes the fluid stored therein (which in the present example is the rehydration liquid) out of the fluid housing (1024) and onto the bottom layer (1026).

The fluid secreted above is delivered from the bottom layer (1026) to the mouth (204) of the user (202) through the plurality of holes (1028) in the bottom layer (1026). If the top layer (1022) has also been provided with the plurality of holes then the fluid is delivered from the top layer (1022) to the palate (2044) as well. The fluid after entering the mouth (204) is mixed with saliva or water to be easily assimilated and provides the required rehydration that the user needs, without having to stop his/her activity. Similarly, other fluids such as nutritional drinks, oxygen etc. may be provided in the fluid housing (1024) and delivered to user (202) on-the-go without halting the activity. For example: if a person is suddenly starts experiencing difficulty in breathing or short breath, then in that case the wearable device (100) with pre-filled oxygen as fluid may save his/her life as the wearable device (100) is easier to carry than an oxygen cylinder.

The present invention offers a number of advantages. Firstly, it is one of a kind solution that has never been tried before to deliver essential drinks to the users on-the-go. Secondly, it can be used as a normal mouth guard too which in itself is a significant improvement as compared to existing mouth guards. The presently available mouth guards or mouth pieces just have a function to protect the teeth, whereas the present invention exceeds that and even has the means to hydrate the user while in use. Existing products allow for the collection and accumulation of excessive saliva while engaging in sporting and athletic events while the present invention utilizes the saliva as a fuel to deliver fluids. Presently existing solutions and mouth guard devices are generally heavy in weight and bulky in design. They also occupy more space in the mouth than necessarily required. Whereas the design of the present invention extracts maximum value out of mouth guard and uses a minimum available space in the mouth. The meticulously designed wearable device of the present invention offers features and benefits of delivering sports nutrition, nutraceuticals, pharmaceuticals, fluids for breathing assistance etc. in a lightweight, easy to use and comfortable wearable product.

The terms and descriptions used herein are set forth by way of illustration only and are not meant as limitations. Examples and limitations disclosed herein are intended to be not limiting in any manner, and modifications may be made without departing from the spirit of the present disclosure. Those skilled in the art will recognize that many variations are possible within the spirit and scope of the disclosure, and their equivalents, in which all terms are to be understood in their broadest possible sense unless otherwise indicated.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the disclosure is anticipated to hold on to all other such alternatives, modifications, and variations that fall within the scope of the present disclosure and appended claims.

1. An intra oral wearable device for delivering fluids, the wearable device comprising:
a central body adapted to be positioned in a mouth of a user, the central body having:
a top layer;
a bottom layer comprising a plurality of holes; and
a fluid housing enclosed between the top layer and the bottom layer, the fluid housing containing a fluid;
one or more tentacles connected at a periphery of the central body; and
one or more inserts adapted to be inserted over the one or more tentacles to improve adherence and comfort;
wherein the one or more inserts and the one or more tentacles are configured to be clamped to an upper jaw of the mouth of the user and secure the central body therein;
wherein after positioning the central body in the mouth, the top layer is proximal to a palate inside the mouth and the bottom layer is proximal to a tongue of the user;
wherein the bottom layer is adapted to be pressed by the tongue that consequently squeezes the fluid housing to secrete the fluid stored therein;
wherein the fluid secreted above is delivered to the mouth of the user through the plurality of holes in the bottom layer.

2. The wearable device as claimed in claim 1, wherein the central body and the tentacles are an integral unit.

3. The wearable device as claimed in claim 1, wherein the central body, the one or more tentacles and the one or more inserts are made of silicone.

4. The wearable device as claimed in claim 1, wherein the fluid is selected from one of nutraceutical fluids, pharmaceutical fluids, re-hydration liquids or gels, nutritional liquids and oxygen.

5. A method for delivering fluids intra orally comprising the steps of:
positioning a central body of a wearable device in a mouth of a user by clamping one or more tentacles to an upper jaw of teeth;
pressing a bottom layer of the central body by a tongue and consequently squeezing a fluid housing to secrete a fluid stored therein;
delivering the fluid secreted to the mouth of the user through a plurality of holes in the bottom layer.

6. The method as claimed in claim 5, wherein the fluid is selected from one of nutraceutical fluids, pharmaceutical fluids, re-hydration liquids or gels, nutritional liquids and oxygen.
